SEQUENCE LISTING (1 sequence)

SEQ ID NO: 1 - protein, 10 aa, Artificial sequence
  optimized helical linker sequence in Trip- cat enzyme-1
HAIMGVAFTW

(19) United States
(12) Patent Application Publication
(10) Pub. No.: US 2011/0086009 A1
(43) Pub. Date: Apr. 14, 2011

(54) Cell line for the production of prostacyclin and uses thereof

(76) Inventor: Ke-He Ruan, Bellaire, TX (US)

(21) Appl. No.: 12/925,088

(22) Filed: Oct. 13, 2010

Related U.S. Application Data
(60) Provisional application No. 61/278,868, filed on Oct. 13, 2009.

(51) Int. Cl. A61K 35/12; C12N 9/00; C12N 9/02; C12N 5/10; C07H 21/00; C12N 15/63; A61P 9/10

(52) U.S. Cl. 424/93.7; 435/183; 435/189; 435/325; 536/23.2; 435/320.1

(57) ABSTRACT

Provided herein are methods for producing a biomolecule by converting or engineering a plurality of cells, e.g., mammalian cells, having an endogenous precursor of a biomolecule to cells transfected to contain a nucleic acid that stably overexpresses a hybrid enzyme effective to metabolized the precursor to the biomolecule in the cell, e.g., prostacyclin produced from arachidonic acid. Also provided are methods of treating a pathophysiological condition associated with at least a decrease in the biomolecule or treating a vascular disease by administering the engineered, stably transfected mammalian cells to a subject where production of the biomolecule treats the condition or vascular disease. Further provided are the recombinant hybrid enzymes and the engineered cell lines and pharmaceutical compositions thereof, the nucleic acids encoding the hybrid enzymes, and vectors comprising the nucleic acids.

### CROSS-REFERENCES TO RELATED APPLICATION

This nonprovisional application claims benefit of priority under 35 U.S.C. §119(e) of provisional U.S. Ser. No. 61/278,868, filed Oct. 13, 2009, now abandoned, the entirety of which is hereby incorporated by reference.

### FEDERAL FUNDING LEGEND

This invention was created in part using funds from the National Institute of Health under Grants HL56712 and HL79389. Consequently, the federal government has certain rights in this invention.

### BACKGROUND OF THE INVENTION

1. Field of the Invention

The present invention relates generally to molecular biology approaches to the commercial production of prostaglandins. More specifically, the present invention relates to the design, construction, and use of COX-1-linker-PGIS cDNA and its transfer into therapeutic cells for the continuous production of prostacyclin (PGI₂).

2. Description of the Related Art

Since the discovery of prostacyclin (PGI₂), there have been ongoing attempts for decades to use prostacyclin and its mimics to treat vascular diseases. Many studies have been reported, in which prostacyclin and its mimics have been used to treat hypertension, atherosclerosis, peripheral vascular diseases, and congestive heart failure. However, one of the major limitations associated with the use of such compounds are their short half life. The present invention provides a novel approach for the production and use of endogenous prostacyclin to protect the vascular system.

A significant amount of evidence has proven that prostacyclin (PGI₂), one of the most important anti-platelet aggregation and vasodilation mediators, plays a key role in the prevention and treatment of vascular and heart diseases. For instance, cyclooxygenase (COX) isoform-2 (COX-2) inhibitors, which suppress prostacyclin biosynthesis in endothelial cells, could be linked to an increase in heart disease as seen in human clinical trials. A study in prostacyclin receptor-knocked out mice also showed an increase in thrombosis tendency. The most popular non-steroidal anti-inflammatory drugs (NSAIDs), which are commonly used for the prevention and treatment of inflammation, may reduce prostacyclin biosynthesis in cells through their inactivation of COX-1 and COX-2, which are the upstream enzymes required for prostacyclin production.

On the other hand, an increase in prostacyclin signaling has been used for treating vascular and heart diseases. For example, the administration of a prostacyclin analog is one of the most widely used treatments for pulmonary artery hypertension (PAH) and has been associated with a reduction in pulmonary artery hypertension-associated mortality. However, due to their short half-life, prostacyclin and its analogs are not capable of achieving long-term pulmonary artery hypertension care. During recent studies, prostacyclin synthase (PGIS) gene transfer could significantly (although temporarily) increase the prostacyclin serum levels and improve hemodynamics in animal models. Nevertheless, the effective and stable biosynthesis of prostacyclin requires an increase in the expression of COX coordinated with PGIS, since COX is a rate-limiting enzyme in prostacyclin biosynthesis. Therefore, the discovery of a system that specifically increases prostacyclin biosynthesis in cells is critical for developing a preventive and therapeutic intervention against vascular and heart diseases.

The prior art is deficient in the lack of a system that specifically increases prostacyclin biosynthesis in cells. The present invention fulfills this longstanding need and desire in the art.

### SUMMARY OF THE INVENTION

The present invention is directed to a method for producing a biomolecule in a cell. The method comprises converting a plurality of cells having an endogenous biosynthetic precursor of a biomolecule into a plurality of mammalian cells and transfecting the cells with a vector containing a nucleic acid that stably overexpresses a hybrid enzyme effective to metabolize the biosynthetic precursor into the biomolecule in the mammalian stable cells. A related invention is the stably transfected mammalian cells produced by this method described herein or a pharmaceutical composition thereof.

The present invention also is directed to a method for treating a pathophysiological condition associated with at least a decrease in production of a biomolecule in a subject. The method comprises administering to the individual a pharmacological amount of the transfected mammalian stable cells described herein such that the mammalian stable cells produce the biomolecule in the subject, thereby treating the pathophysiological condition.

The present invention is directed further to a method for producing prostacyclin in a cell. The transfected mammalian cells have a vector containing a cDNA that stably overexpresses a hybrid enzyme effective to metabolize the arachidonic acid into prostacyclin in the mammalian stable cells.

The present invention is directed further still to a cell line engineered to produce prostacyclin. The cell line having endogenous arachidonic acid transformed with a nucleic acid that stably overexpresses a human hybrid enzyme effective to metabolize the arachidonic acid into prostacyclin in the engineered cells. The present invention is directed to a related invention that is a pharmaceutical composition comprising a plurality of the engineered cells described herein where the hybrid enzyme has a human cyclooxygenase isoform-1 or -2 enzyme and a human prostacyclin synthase enzyme linked with the sequence shown in SEQ ID NO: 1 and a pharmaceutically acceptable carrier.

The present invention is directed further still to a method for treating a vascular disease in a subject. The method comprises administering to the individual a pharmacologically effective amount of the engineered cells described herein where the engineered cells produce prostacyclin in the subject thereby treating the vascular disease.

The present invention is directed further still to a recombinant human hybrid protein. The hybrid enzyme comprises a human cyclooxygenase isoform-1 or -2 enzyme, a human prostacyclin synthase enzyme and a helical amino acid sequence with about 10 residues linking said enzymes. Most preferably, the helical amino acid sequence is shown in SEQ ID NO: 1.

The present invention is directed further still to a nucleic acid that encodes a human hybrid protein. The nucleic acid may comprise a) an isolated nucleic acid encoding the human hybrid protein described herein, b) an isolated nucleic acid that hybridizes to isolated DNA of a) above and c) an isolated nucleic acid differing from the isolated nucleic acid of a) and b) above in codon sequence due to the degeneracy of the genetic code that encodes the human hybrid protein. The present invention is directed to a related invention that is a vector comprising the nucleic acid described herein and regulatory elements necessary to stably overexpress the nucleic acid in a cell.

Other and further aspects, features, and advantages of the present invention will be apparent from the following description of the presently preferred embodiments of the invention. These embodiments are given for the purpose of disclosure.

### BRIEF DESCRIPTION OF THE DRAWING

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows the time-course of the differentiation of mouse adipocytes under a conditional medium using the method described. The adipocytes, with lipid droplets, are collected from mouse adipose tissue and placed between two cover slides (Day 1). The cells are cultured using the conditional medium described herein. The days of the culture are indicated on the labels.

FIG. 2 shows the determination of the [¹⁴C] arachidonic acid metabolism in the endothelial-like fat cells using an isotope-HPLC method. Briefly, the cells (˜0.1×10⁶) are washed and then incubated with [¹⁴C] arachidonic acid (10 μM) for five minutes. The metabolized [¹⁴C] eicosanoids from the [¹⁴C] arachidonic acid in the supernatant are analyzed by HPLC on a C18 column (4.5×250 mm) connected to a liquid scintillation analyzer.

FIGS. 3A-3B show profiles of the Western blot analyses for prostanoids' synthesizing enzymes (FIG. 3A) and receptors (FIG. 3B) for the endothelial-like fat cells. The cells (1×10⁶) were separated by 10% SDS-PAGE and then transferred to a nitrocellulose membrane. The endogenous prostanoids' enzymes and receptors are stained by respective antibodies. The COX-1, PGIS, cPGES and TXAS are identified in FIG. 3A, and the IP, TP, EP1, EP2, EP3 and EP4 receptors are identified in FIG. 3B. The inducible COX-2 and mPGES-1 are not present (FIG. 3A).

FIGS. 4A-4B show a western blot analysis and [¹⁴C] arachidonic acid metabolism, respectively. FIG. 4A shows a Western blot analysis for the transiently transfected endothelial-like fat cells using 7% SDS-PAGE. Untransfected endothelial-like fat cells (1) and those endothelial-like fat cells transiently expressing COX-1-10aa-PGIS (2) are shown. The Trip-cat enzyme-1 is indicated with an arrow. FIG. 4B shows the determination of the [¹⁴C] arachidonic acid metabolism in the transiently transfected endothelial-like fat cells. The metabolized [¹⁴C] eicosanoids are determined in the transiently transfected endothelial-like fat cells (˜0.3×10⁶) using the same procedure as that described in FIG. 2.

FIGS. 5A-5B show an analysis of PGI₂-endothelial-like fat cells. FIG. 5A shows a Western blot analysis and activity assay for the PGI₂-endothelial-like fat cells. The proteins of the cultured cells are separated by 7% (w/v) SDS-PAGE under denaturing conditions and then transferred to a nitrocellulose membrane. Bands recognized by anti-PGIS primary antibody are visualized with horseradish peroxidase-conjugated secondary antibody. Untransfected endothelial-like fat cells (1) and the PGI₂-endothelial-like fat cells (stably expressing COX-1-10aa-PGIS (2)) are shown using the same conditions to that described in FIG. 4A. FIG. 5B shows a determination of the [¹⁴C] arachidonic acid metabolism in the PGI₂-endothelial-like fat cells. The metabolized [¹⁴C] eicosanoids are determined in PGI₂-endothelial-like fat cells (˜0.1×10⁶) using the same procedure described in FIG. 2.

FIG. 6A shows data relating to PGI₂-production. FIG. 6A shows the time course of PGI₂-production in the endothelial-like fat cells stably expressing COX-1-10aa-PGIS (circles) and co-expressing the parent enzymes, COX-1 and PGIS (squares). The results are normalized and shown as percent activity, based on the levels of prostacyclin production. The assay and HPLC analysis conditions used are similar to those described in FIG. 2, and only the reaction times are varied. FIG. 6B shows the activity of the transient (white squares) and the stable (black squares) expression of COX-1-10aa-PGIS in the endothelial-like fat cells, according to the amount of [¹⁴C] 6-keto-PGF_{1α} (degraded PGI₂). Each group of cells was taken for assay analysis at different days following the transfection. The assay conditions are described in FIG. 2.

FIG. 7 shows the effects of the PGI₂-endothelial-like fat cells on platelet aggregation. The platelet-rich plasma (450 μL per sample) is incubated with 50 μL PBS (A), 0.1×10⁶ endothelial-like fat cells (B), or 0.1×10⁶ PGI₂-ELFCs (C) for 1 minute, and then the platelet aggregation is initiated by the addition of 50 μL sodium arachidonate (5 mg/mL) into the plasma sample. The data are collected from monitoring the platelet aggregation.

FIGS. 8A-8H shows the effects of the PGI₂-ELFCs on reperfusion of the mouse hindlimb-ischemic model as monitored by LDPI. The blood flow images (FIGS. 8A-8D) and reperfusion intensity (FIGS. 8E-8H) of the mouse hindlimbs with (FIGS. 8B-8D and 8F-8H) and without (FIGS. 8A, 8E) femoral artery ligation in the right leg are displayed. The red color within the circled areas (FIGS. 8A-8D) in both hindlimbs represents the blood flow intensity (perfusion), which is integrated and plotted, as shown in FIGS. 8E-8H. Normal blood perfusion in both the left (“L”) and right (“R”) mouse hindlimbs are shown in FIGS. 8A and 8E. The perfusion after injection of PBS (FIGS. 8B, 8F), PGI₂-ELFCs (FIG. 8C, 8G), or untransfected endothelial—like fat cells (FIGS. 8D, 8H) in the femoral artery-ligated right hindlimb are shown (n=3).

### DESCRIPTION OF THE PREFERRED EMBODIMENTS

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “or” in the claims refers to “and/or” unless explicitly indicated to refer to alternatives only or the alternatives are mutually exclusive, although the disclosure supports a definition that refers to only alternatives and “and/or”.

As used herein “hybrid enzyme” refers to an enzyme engineered to contain al or elements of two or more enzymes. The component enzymes may be joined or linked with an amino acid sequence that allows for optimal catalytic activities of the separate enzymes or elements thereof comprising the hybrid enzyme.

As used herein, the term “subject” refers to any recipient of the engineered cells and/or the biomolecule produced thereby.

The following abbreviations used herein are: AA, arachidonic acid; COX-1, cyclooxygenase isoform-1; COX-2, cyclooxygenase isoform-2; cPGES, cytosolic prostaglandin E₂ synthase; ELFC, endothelial—like fat cell; EP1-4, prostaglandin E₂ receptor subtypes 1-4; HPLC, high-performance liquid chromatography; IP, Prostacyclin receptor; LDPI, laser doppler perfusion imager; mPGES, microsomal prostaglandin E₂ synthase; PCR, polymerase chain reaction; PGE₂, prostaglandin E₂; PGES, prostaglandin E₂ synthase; PGI₂, prostaglandin I₂ (prostacyclin); PGIS, prostacyclin synthase; Trip-Cat, Triple-catalytic; TP, Thromboxane A₂ receptor; TXA₂, Thromboxane A₂; TXAS, Thromboxane A₂ synthase.

In one embodiment of the present invention there is provided a method for producing a biomolecule in a cell, comprising transfecting a plurality of mammalian cells with a vector containing a nucleic acid that stably overexpresses a hybrid enzyme effective to metabolize the biosynthetic precursor into the biomolecule of interest in the stably transfected mammalian stable cells.

In this embodiment the hybrid enzyme may comprise two or more enzymes linked together that metabolize the biosynthetic precursor and its products to produce the biomolecule. Also, the hybrid enzymes may comprise human enzymes. For example, the hybrid enzyme may be a human cyclooxygenase isoform-1 or -2 enzyme, a human prostacyclin synthase enzyme and a helical amino acid sequence linking the enzymes. Particularly, the helical amino acid sequence is that shown in SEQ ID NO: 1. The human hybrid enzyme may be Trip-cat enzyme-1. In addition, the vector may be a non-viral vector. Furthermore, the stably transfected mammalian cells may produce the biomolecule continuously. In an aspect of this embodiment the endogenous biosynthetic precursor is arachidonic acid and the biomolecule is prostacyclin.

In a related embodiment there is provided stably transfected mammalian cells produced by the methods described supra or a pharmaceutical composition thereof.

In another embodiment of the present invention there is provided a method for treating a pathophysiological condition associated with at least a decrease in production of a biomolecule in a subject, comprising administering to the subject a pharmacological amount of the stably transfected mammalian cells described supra, where the mammalian cells produce the biomolecule in the subject, thereby treating the pathophysiological condition. In this embodiment the stably transfected mammalian cells may produce the biomolecule continuously. Examples of the pathophysiological condition are hypertension, atherosclerosis, peripheral vascular diseases, or congestive heart failure.

In yet another embodiment of the present invention there is provided a method for producing prostacyclin in a cell, comprising transfecting a plurality of mammalian cells with a vector containing a nucleic acid that stably overexpresses a hybrid enzyme effective to metabolize the arachidonic acid to prostacyclin in the mammalian stable cells. In this embodiment the vector, the hybrid enzyme and the continuous production of the biomolecule are as described supra.

In yet another embodiment of the present invention there is provided a cell line engineered to produce prostacyclin, comprising a plurality of mammalian cells having endogenous arachidonic acid transformed with a nucleic acid stably overexpressing a human hybrid enzyme effective to metabolize the arachidonic acid to prostacyclin in the engineered cells. In this embodiment the cells may produce prostacyclin continuously. Also, the vector and the hybrid enzyme are as described supra.

In a related embodiment the present invention provides a pharmaceutical composition, comprising a plurality of the engineered cells described supra where the hybrid enzyme comprises a human cyclooxygenase isoform-1 or -2 enzyme and a human prostacyclin synthase enzyme linked with the sequence shown in SEQ ID NO: 1; and a pharmaceutically acceptable carrier.

In yet another embodiment of the present invention there is provided a method for treating a vascular disease in a subject, comprising administering to the subject a pharmacologically effective amount of the engineered cells described supra, where the engineered cells produce prostacyclin in the subject thereby treating the vascular disease. In this embodiment the vascular disease may be hypertension, atherosclerosis, peripheral vascular diseases, or congestive heart failure. Also, the engineered cells may produce prostacyclin continuously.

In yet another embodiment of the present invention there is provided a recombinant human hybrid protein, comprising a human cyclooxygenase isoform-1 or -2 enzyme; a human prostacyclin synthase enzyme; and a helical amino acid sequence with about 10 residues linking the enzymes. In this embodiment the helical linker may have a sequence shown in SEQ ID NO: 1. Most preferably, the human hybrid protein is Trip-cat enzyme-1 or -2.

In yet another embodiment of the present invention there is provided a nucleic acid encoding a human hybrid protein, comprising: a) an isolated nucleic acid encoding the human hybrid protein described supra; b) an isolated nucleic acid that hybridizes to isolated DNA of a) above; and c) an isolated nucleic acid differing from the isolated nucleic acid of a) and b) above in codon sequence due to the degeneracy of the genetic code that encodes the human hybrid protein.

In a related embodiment the present invention provides a vector comprising the nucleic acid described supra and regulatory elements necessary to stably overexpress said nucleic acid in a cell. In this embodiment the vector may be a non-viral vector.

The present invention describes the specific biosynthesis of prostacyclin in cells through engineering of a novel enzyme which linked cycloloxygenase isoform-1 or -2 (COX-1, COX-2) to prostacyclin synthase enzyme (PGIS), and contains unique triple-catalytic (Trip-cat) functions to effectively convert arachidonic acid (AA) into prostacyclin. As such, the invention described herein relates to the design, construction and use of a cell line of primary cultured mammalian cells that constantly synthesize prostacyclin and continue to grow for a long period of time, thereby demonstrating therapeutic potential against vascular and heart diseases.

This invention builds on the integration of two approaches, namely the engineering of a novel Trip-cat enzyme by linking COX-1 to PGIS through an optimized sequence. Such integration leads to a stable expression system that optimizes the cell culture conditions for prostacyclin biosynthesis. This present invention is a model for the engineering of PGI₂-producing cells derived from other cell types, such as stem cells and stromal cells. It also provides a basis for using a membrane protein as a therapeutic reagent through cell engineering and delivery strategies.

For instance, adipocytes isolated from mouse adipose tissue were cultured and converted into “Endothelial-like Fat Cells” that expressed endogenous cyclooxygenase-1 (COX-1), prostacyclin synthase (PGIS), cytosolic prostaglandin E₂ (PGE₂) synthase (cPGES), and the corresponding receptors for PGI₂, thromboxane A₂ (TXA₂), and PGE₂. The endothelial—like fat cells, which essentially metabolize endogenous arachidonic acid (AA) into PGE₂ (a pro-inflammatory mediator), were re-engineered through gene transfection of a newly constructed human hybrid enzyme cDNA (linked COX-1 and PGIS cDNAs) using a non-viral vector and G418 screening to generate a stable cell line (PGI₂-ELFCs). The gene transfer shifts the arachidonic acid metabolism products from PGE₂ (in the untransfected endothelial—like fat cells) to prostacyclin (in the transfected endothelial—like fat cells) with a PGI₂/PGE₂ ratio change from 0.03 to 25.

The PGI₂-endothelial—like fat cells, which exhibited approximately a 50-fold increase in prostacyclin biosynthesis, demonstrated superior anti-platelet aggregation activities in vitro and increased reperfusion in the mouse ischemic hindlimb model in vivo. An advantage of this engineered cell line is the continuous or steady production of prostacyclin. It is therefore contemplated that the engineered cell lines described herein may be produced for commercial applications, such as production of prostacyclin. It also provides a model for finding a novel therapeutic approach by way of integrated gene, cell, and protein engineering.

Thus, the present invention provides methods of treating pathophysiological conditions, such as, but not limited to, vascular or heart diseases, e.g., hypertension, atherosclerosis, peripheral vascular diseases, or congestive heart failure. For example, the engineered mammalian cells and/or the prostacyclin they produce may be administered to a subject in need of such treatment. The present invention also provides pharmaceutical compositions comprising a plurality of the engineered mammalian cells which stably overexpress the hybrid enzyme comprising a human cyclooxygenase isoform-1 or -2 enzyme and a human prostacyclin synthase enzyme linked with the sequence shown in SEQ ID NO: 1. The pharmaceutical compositions may comprise a pharmaceutically acceptable carrier, adjuvant or diluent as are known and standard in the art.

One ordinary skill in the art is well suited to determine therapeutically effective methods of delivery or administration of one or both of the engineered mammalian cells or the prostacyclin produced thereby, including dose and dosage schedule. As is known in the art, such determinations depend on the subject's age, sex, weight, health, type and progression of the disease, etc. Upon administration or delivery to the subject, the engineered mammalian cell lines described herein advantageously provide continuous or steady prostacyclin to the subject over an extended period of time.

It is contemplated that the gene, cellular and protein engineering methods and procedures described herein can be extended to other cell types and enzymes. Generally, a cell having an endogenous precursor for a biomolecule can be transfected with a suitable vector encoding a nucleic acid or cDNA that is engineered to stably and continuously overexpress a hybrid enzyme. The hybrid enzyme metabolizes the precursor to produce the desired biomolecule. As described herein, these engineered cells and/or biomolecules produced thereby may be useful as therapeutics or may provide the basis for commercial production. For example, as therapeutics the engineered cells and/or produced biomolecules may be used to treat pathophysiological conditions associated with at least a decrease in the level or amount of biomolecule produced in a subject.

The following examples are given for the purpose of illustrating various embodiments of the invention and are not meant to limit the present invention in any fashion.

### Example I

### Materials

Medium for culturing the cell lines was purchased from Invitrogen. [¹⁴C] arachidonic acid was purchased from Amersham (Piscataway, N.J.). Collagenase and general chemicals are purchased from Sigma (St. Louis, Mo.). Arachidonic Acid and primary antibodies against human COX-1, COX-2, PGIS, cPGES, mPGES, TP, IP, and the four EP receptors (EP1-4) are purchased from Cayman Chemicals.

### Example 2

### Engineered cDNA Plasmids with Single Genes Encoding the Human COX-1 and PGIS Sequences

The sequence of COX-1 linked to prostacyclin synthase through a 10 amino acid (10aa) linker (COX-1-10aa-PGIS, Trip-cat enzyme-1) was generated by a PCR approach and subcloning procedures provided by the vector company (Invitrogen). Briefly, the corresponding cDNA sequence was isolated from the pSG5 vector containing human COX-1 or PGIS by PCR and the cDNA is further subcloned into the pcDNA3.1 vector to form pcDNA3.1-COX-1-10aa-prostacyclin synthase.

### Example 3

### Establishing a Primary Cultured Cell Line Derived from Mouse Adipose Tissue

Eight-to-ten-week old male C57BU6J mice were purchased from Harlan Sprague Dawley (Indianapolis, Ind.). Briefly, inguinal adipose tissue is excised from a mouse and washed thoroughly with saline before undergoing digestion by collagenase (2 mg/mL) at 37° C. for one hour. The digested adipose tissue is then filtered through glass wool and subjected to low speed centrifugation for further separation. The mouse adipocytes (MA) collected from the upper-layer of the collagenase-digested mouse adipose tissue are placed between two cover slides in a 10-cm cell culture dish with DMEM-F12 medium containing 10% fetal bovine serum and 1% antibiotic and antimycotic, and then are grown at 37° C. in a humidified 5% CO₂ incubator.

### Example 4

### Transient and Stable Expression of the Trip-Cat Enzyme-1 in the ELFCs

The recombinant Trip-cat enzyme-1 was expressed in the cultured cells using a similar method as described. Briefly, the cells were grown and transfected with the purified pcDNA3.1-COX-1-10aa-PGIS by the Lipofectamine 2000 method following the manufacturer's instructions (Invitrogen). For the transient expression, the cells were harvested approximately 48 h after transfection for further enzyme assays and Western blot analysis. For the stable expression, the transfected cells were cultured in the presence of geneticin (G418 screening) for several weeks following the manufacturer's instructions. The cells stably expressing the Trip-cat enzyme-1 are identified by enzyme assay and Western blot analysis.

### Example 5

### Enzyme Activity Determination for the Trip-Cat Enzyme-1 Using the HPLC Method

To determine the activities of the synthases that converted arachidonic acid to prostacyclin through the triple catalytic functions, different concentrations of [¹⁴C] arachidonic acid are added to the PGI₂-ELFCs in a total reaction volume of 100 μL. After 10 secondes to 15 minutes of incubation, the reactions are stopped by adding 200 μL of the solvent (solvent A) containing 0.1% acetic acid and 35% acetonitrile. After centrifugation (12,000 rpm for 5 minutes), the supernatant is injected into a C18 column (Varian Microsorb-MV 100-5, 4.6×250 mm) using the solvent A with a gradient from 35 to 100% of acetonitrile for 45 minutes at a flow-rate of 1.0 mL/min. The [¹⁴C] labeled arachidonic acid metabolites, including 6-keto-PGF_{1α} (degraded PGI₂) were monitored directly by a flow scintillation analyzer.

### Example 6

### Determination of the Endogenous AA Metabolisms in the Cultured Cells Using LC/MS

Simultaneous quantification of arachidonic acid metabolites in the cultured cells was detected by LC/MS following the reported methods. Briefly, the cell culture media was collected and applied to a C18 cartridge. After washing with water, the arachidonic acid metabolites bound to the cartridge were eluted with acetone, dried by nitrogen gas, and then dissolved in solvent A. The sample was injected into the Waters Micromass LC/MS/MS system by an auto-sampler. The metabolites were first separated by the RP-HPLC C18 column and then automatically injected into the mass detector equipped with an ESI source in a negative mode. Synthetic arachidonic acid, PGE₂, and 6-keto-PGF_{iα}, obtained from Cayman Chemical Company (Ann Arbor, Mich.), were used as standards to calibrate the LC/MS system, identify the corresponding prostanoids, and normalize the detection limits and sensitivities.

### Example 7

### Immunoblot Analysis

The cultured cells were collected and washed with PBS. The proteins are separated by 7-10% (w/v) SDS-PAGE under denaturing conditions and then transferred to a nitrocellulose membrane. Bands recognized by individual primary antibodies are visualized with horseradish peroxidase-conjugated secondary antibody as described.¹⁴

### Example 8

### Anti-Platelet Aggregation Assays

A sample of fresh blood was collected using a blood collection tube with 3.2% sodium citrate for anticoagulation, and centrifuged to separate the plasma from the red blood cells. A total of 450 μL of this platelet rich plasma was placed inside the 37° C. incubator of an aggregometer (Chrono-Log) for a total of 3 minutes. 50 μL of sodium arachidonate (5 mg/mL) is added to the platelet rich plasma in the absence and presence of the cultured cells. The platelet aggregation is then monitored.

### Example 9

### Mouse Hindlimb Ischemia Model

The mice are anesthetized with 50 mg/kg pentobarbital (given intraperitoneally). Excess hairs were removed by depilatory cream from both hindlimbs, and the proximal portion of the femoral artery (right side) is ligated with a silk suture. The overlying skin was closed with three surgical staples. The mice are then randomized to three groups: (i) for PBS injection; (ii) for PGI₂-endothelial—like fat cells injection and (iii) for non-transfected endothelial—like fat cell injection. After 24 hours of the femoral artery ligation, PBS, endothelial—like fat cells (1.5×10⁶ cells) or PGI₂-endothelial—like fat cells (1.5×10⁶ cells) were injected subcutaneously at 3-different sites around the ischemic region. The effects of the PGI₂-endothelial—like fat cells and non-transfected endothelial—like fat cells on blood reperfusion in the ischemic hindlimb were measured after 48 hours of the injection, using a Laser Doppler Perfusion Imager System (LDPI). Once the laser doppler color images are recorded three times, the average perfusion of the ischemic and non-ischemic hindlimbs is calculated on the basis of colored histogram pixels.

### Example 10

### Isolation, Culture, and Conversion of the Adipocytes into Endothelial-Like Fat Cells

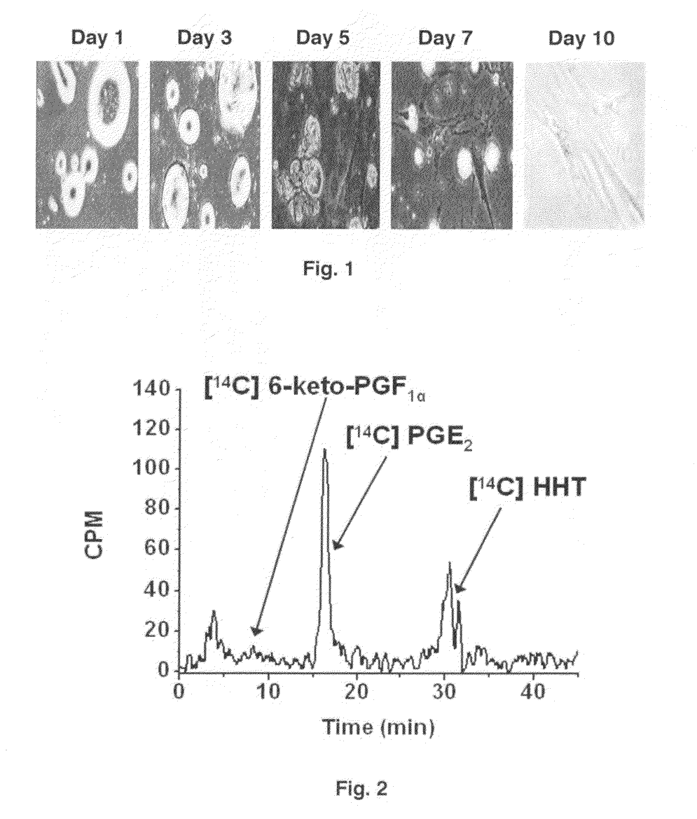

FIG. 1 shows the time course of the differentiation of the adipocytes under the conditional medium. The lipid droplets within the adipocytes, placed between the cover slides, gradually disappear by the time the cells are first cultured. After removal of the cover slides, the lipid-free cells began to grow onto the plate, while retaining their morphological shape (FIG. 1, Day 10). The cells are able to metabolize endogenous arachidonic acid and synthesize a large amount of PGE₂ (3 ng/10-cm dish cells) and a very small amount of 6-keto-PGF_{iα}(0.1 ng/10-cm dish cells), as identified by LC/MS analysis (Table 1).

TABLE 1
Comparison of the biosynthesis ratio of the endogenous prostacyclin and
PGE₂ in the ELFCs and PGI₂-ELFCs as determined by LC/MS

| | PGI₂ | PGE₂ | |
|---|---|---|---|
| | (ng/10-cm | (ng/10-cm | |
| | dish of cells) | dish of cells) | PGI₂/ |
| Cell Types | (n = 3) | (n = 3) | PGE₂ |
| ELFC | 0.1 ± 0.01 | 3 ± 0.1 | 0.03 |
| PGI₂-ELFC | 5 ± 0.3 | 0.2 ± 0.02 | 25 |
| Change in | 50-fold PGI₂ Increase | 15-fold PGE₂ Decrease | N/A |
| Vascular | | | |
| Protection | | | |
| Factors | | | |

To further confirm the arachidonic acid metabolism in the cells, an exogenous arachidonic acid-metabolized profile was investigated by an assay using [¹⁴C] arachidonic acid as a starting substrate as illustrated in FIG. 2. The assay clearly show that [¹⁴C] PGE₂ is a major product of the added [¹⁴C] arachidonic acid (FIG. 2), whereas very little [¹⁴C] 6-keto-PGF_{1α}was observed. This indicated that these cells, similar to the endothelial cells, contain the enzymes necessary to synthesize PGE₂ and PGI₂.

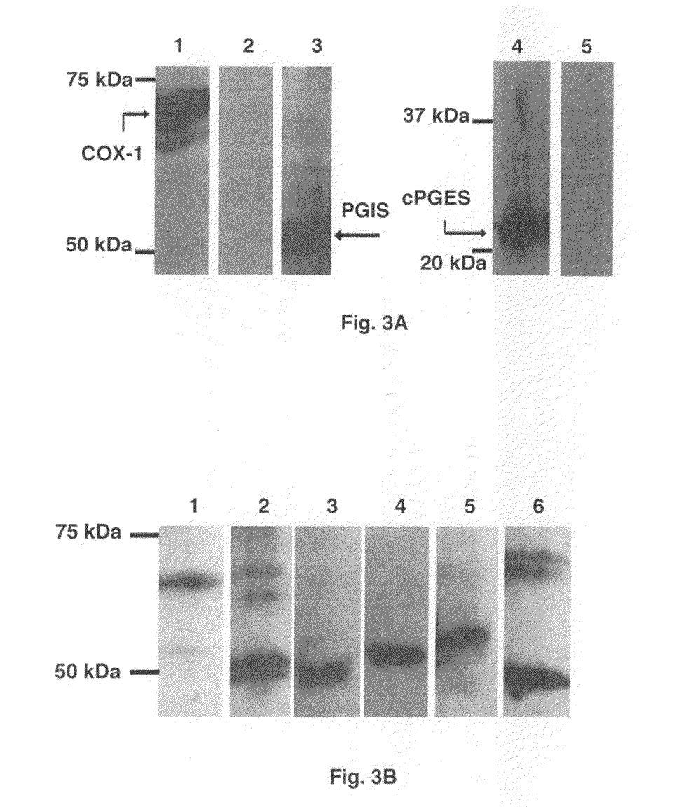

Using Western blot analysis, the cells' expression of COX-1, PGIS, and cPGES was confirmed. The inducible COX-2 and mPGES-1 (FIG. 3A) were not detectable. In addition, the Western blot analysis also reveals that the cells express the TXA₂ receptor (TP), prostacyclin receptor (IP), and the four PGE₂ subtype receptors (EP1, EP2, EP3 and EP4) (FIG. 3B). In addition, the ability to be reproduced for many passages (a stromal cell property) was also confirmed in the cells. Thus, the cells from this cell line are referred to as, “Endothelial-like Fat Cells” (ELFCs).

### Example 11

### Construction of COX-1-10aa-PGIS cDNA and Transient Expression of the COX-1-10aa-PGIS in the ELFCs Using a Non-Viral Vector Approach

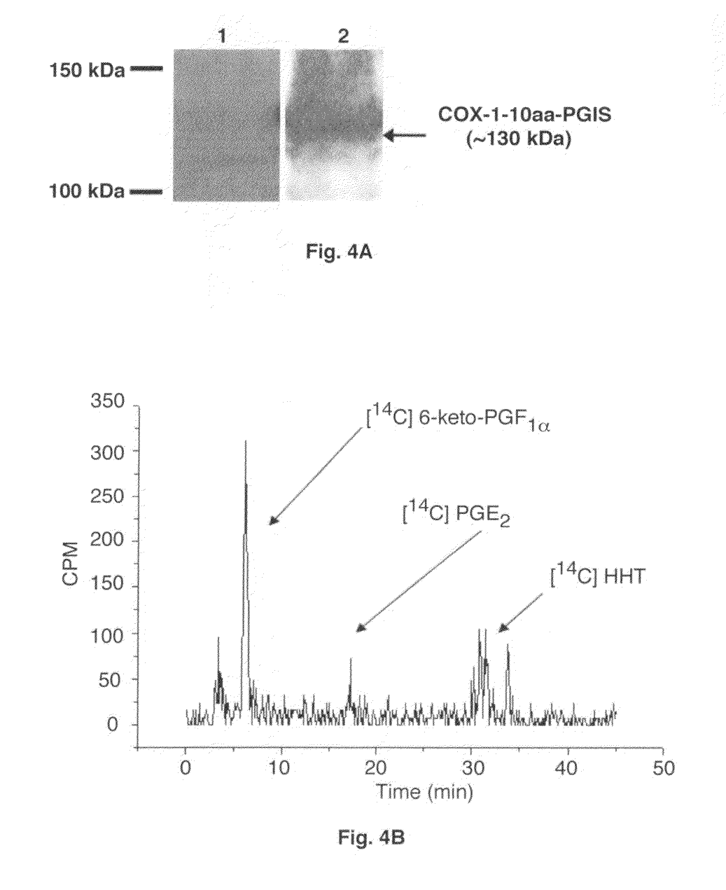

A cDNA sequence encoding a novel Trip-cat Enzyme-1 (COX-1-10aa-PGIS), linking the C-terminus of human COX-1 to the N-terminus of human PGIS by a helical linker with 10 residues (His-Ala-IIe-Met-Gly-Val-Ala-Phe-Thr-Trp (SEQ ID NO: 1, 10aa)) derived from human rhodopsin, was created by a PCR approach and then subcloned into a non-viral vector, pcDNA3.1, with a CMV promotor using a similar approach described. The endothelial-like fat cells were suitable for being engineered into PGI₂-producing cells because these cells contain endogenous arachidonic acid, which can be metabolized by the overexpressed PGI₂-producing enzyme. The endothelial-like fat cells were transfected by the Lipofectamine 2000 method, using the pcDNA3.1-COX-1-10aa-PGIS plasmid. Subsequently, the successful overexpression of the recombinant COX-1-10aa-PGIS protein in the endothelial-like fat cells is confirmed by a Western blot analysis that show the correct molecular mass of the PGI₂-producing enzyme, approximately 130 kDa (FIG. 4A). The transfected cells overexpressing the active COX-1-10aa-PGIS were confirmed by the metabolism of the [¹⁴C] AA into ^{[14}C] 6-keto-PGF_{1α}(degraded [¹⁴C] PGI₂, FIG. 4B). The conversion of endogenous arachidonic acid into prostacyclin by the cells was also observed by LC/MS analysis using the negative mode (data not shown).

### Example 12

Establishing an ELFC Cell Line that can Stably Express COX-1-10aa-PGIS and Constantly Produce PGI₂

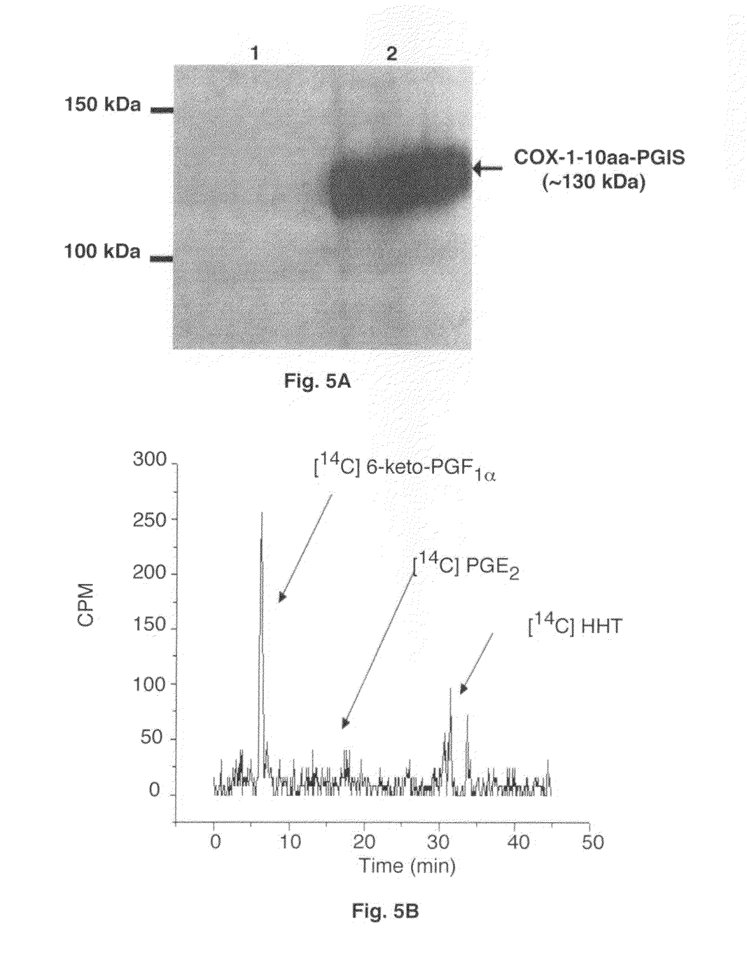

To develop a cell line with therapeutic potential, that can up-regulate prostacyclin biosynthesis, it is necessary to create a method for generating a stable cell line that would overexpress the COX-1-10aa-PGIS. However, very little studies have been done for transformed endothelial-like fat cells which can stably express a recombinant protein. Studies described herein begin with the transient expression of the enzyme described above. After transferring the pcDNA-COX-1-10aa-PGIS into the endothelial-like fat cells, a G418 screening approach was followed to generate the stable cell line. The transient transfection rate for these cells is approximately 5%, which is the result of the cells that remained alive during the G418 screening. After three weeks, the cells stably expressing the COX-1-10aa-PGIS were identified by Western blot analysis (FIG. 5A).

The amount of the COX-1-10aa-PGIS expressed in the stable cell line (FIG. 5A) significantly increased compared to that of its transient expression (FIG. 4A). The endothelial-like fat cells stably expressing COX-1-10aa-PGIS, which effectively metabolized exogenous [¹⁴C] arachidonic acid into [¹⁴C] 6-keto-PGF_{1α}(FIG. 5B), and endogenous arachidonic acid into 6-keto-PGF₁₀ (Table 1), were identified by enzyme assays and LC/MS analyses. By transfecting the COX-1-10aa-PGIS cDNA into the endothelial-like fat cells, the vascular protector, PGI₂, production is increased by 50-fold and the inflammatory PGE₂ synthesis is decreased by 15-fold (Table 1). Thus, the cells resulting from this cell line are termed, PGI₂-endothelial-like fat cells in respect to their specific biosynthesis of PGI₂.

### Example 13

### Kinetic Studies for the Cells Expressing COX-1-10aa-PGIS

The Km and Vmax values for the COX-1-10-aa-PGIS expressed in cells has been characterized by an assay using increasing amounts of [¹⁴C] AA. The determined Km and Vmax values for the expressed COX-1-10aa-PGIS were 5 μM and 400 μM, respectively, which are identical to that of the co-expressed individual COX-1 and PGIS in the cells. The results provided strong evidence to show that the engineered COX-1-10aa-PGIS can integrate the Trip-cat biological functions of the individual COX-1 and PGIS anchors in the ER membrane in mammalian cells.

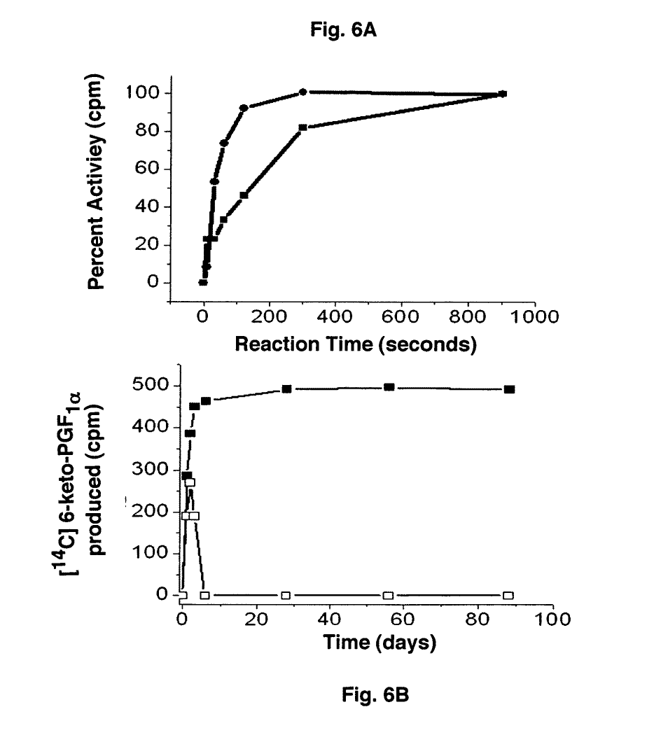

To further characterize the enzyme kinetics, a time course study was also performed to investigate the dynamic changes in the PGI₂-production yield in the PGI₂-ELFCs, in which only 50 seconds are required to generate a 50% production of prostacyclin in the PGI₂-ELFCs (FIG. 6A, circles), whereas approximately 150 seconds were required to generate the same amount of prostacyclin using the co-expressed COX-1 and PGIS (FIG. 6A, squares). This indicated that the COX-1-10aa-PGIS has a better turnover rate compared to that of the mixture of the individual enzymes, COX-1 and PGIS. The effects of the transient (open circles, FIG. 6B) and stable (closed circles, FIG. 6B) expression of the COX-1-10aa-PGIS in the endothelial-like fat cells on the production of prostacyclin are also monitored and compared for 12 weeks. The highest prostacyclin production is recorded at 48 hours and the curve gradually returns to the basal level for the transient expression. However, the stable expression is able to maintain a constant level of prostacyclin biosynthesis for up to three months without any signs of decreasing (FIG. 6B). This indicates that the gene, COX-1-10aa-PGIS, can be passed to several generations of the endothelial-like fat cells, which can be very useful for the delivery of a stable cell line in vivo.

### Example 14

### PGI₂-ELFCs Inhibiting Platelet Aggregation In Vitro

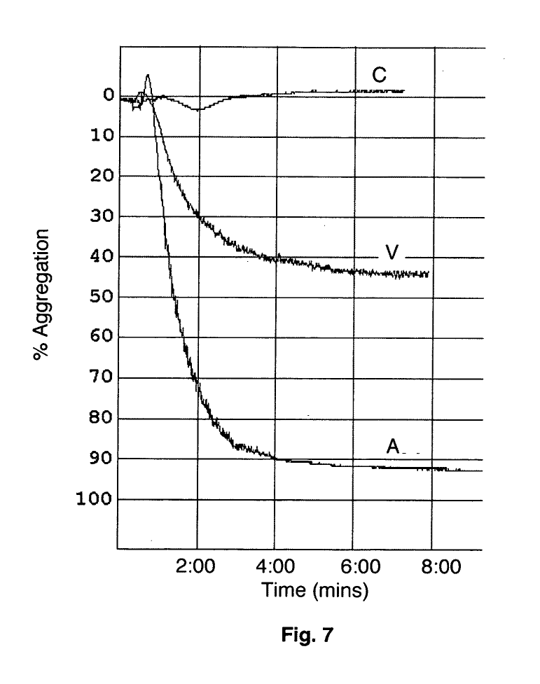

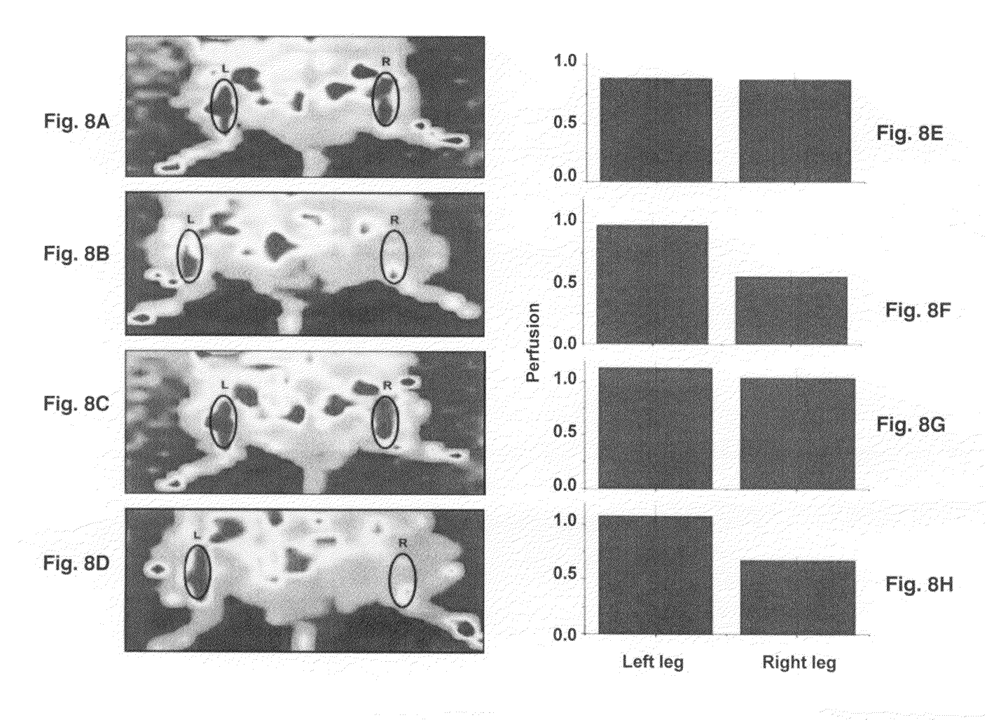

It is known that platelets primarily convert arachidonic acid into TXA₂ through the coupling actions of COX-1 and TXAS. In the platelet rich plasma, the majority of the added arachidonic acid is metabolized into TXA₂, which triggered platelet aggregation in minutes (FIG. 7A). However, in the presence of the PGI₂-ELFCs, the platelet aggregation was completely blocked (FIG. 7C), which indicated that the cells are able to shift the arachidonic acid metabolisms from TXA₂ to prostacyclin and thereby mediate anti-platelet aggregation in the platelet rich plasma. On the other hand, the untransfected endothelial-like fat cells, which produce PGE₂, could only delay, but not completely block the platelet aggregation (FIG. 8B). This indicates that the engineered COX-1-10aa-PGIS, expressed in the endothelial-like fat cells, is able to effectively compete in the arachidonic acid metabolism with the endogenous TXAS. Furthermore, the results imply that the PGI₂-ELFCs should be able to increase prostacyclin levels and decrease TXA₂ production in the circulation for vascular protection if the cells are delivered into the circulation.

### Example 15

Effect of the PGI₂-ELFCs on Mouse Ischemic Hindlimb Model

To test the vascular protection of the PGI₂-endothelial-like fat cells in vivo, a mouse ischemic hindlimb model was generated by placing a ligature on the right femoral artery of the mice, following the procedures described previously. The reduction in blood flow in the right hindlimb, following the ligation, is clearly detected (FIGS. 8B and 8F) using laser a Laser Doppler Perfusion Imager System (LDPI, Perimed, Stockholm, Sweden) compared to that of the unligated right hindlimb (FIGS. 8A and 8E). A single injection of the PGI₂-endothelial-like fat cells at the ischemic hindlimb (right side) shows a significant increase in blood reperfusion as illustrated in FIGS. 8C and 8G). In contrast, the injection of untransfected endothelial-like fat cells could not significantly improve the ischemic condition (FIGS. 8D and 8H). These studies further validate the therapeutic potential of the PGI₂-endothelial-like fat cells used for ischemic protection and treatment.

The present invention is well adapted to attain the ends and advantages mentioned as well as those that are inherent therein. The particular embodiments, objects, ends, and advantages disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope of the present invention.

What is claimed is:

1. A method for producing a biomolecule in a cell, comprising:
transfecting a plurality of mammalian cells with a vector containing a nucleic acid that stably overexpresses a hybrid enzyme effective to metabolize the biosynthetic precursor to the biomolecule in the stably transfected mammalian cells.

2. The method of claim 1, wherein the hybrid enzyme comprises two or more enzymes linked together that metabolize the biosynthetic precursor and its products to produce the biomolecule.

3. The method of claim 1, wherein the hybrid enzymes comprises human enzymes.

4. The method of claim 3, wherein the human hybrid enzyme comprises:
a human cyclooxygenase isoform-1 or -2 enzyme;
a human prostacyclin synthase enzyme; and
a helical amino acid sequence linking said enzymes.

5. The method of claim 4, wherein the helical amino acid sequence is shown in SEQ ID NO: 1.

6. The method of claim 5, wherein the human hybrid enzyme is Trip-cat enzyme-1 or -2.

7. The method of claim 1, wherein the endogenous biosynthetic precursor is arachidonic acid and the biomolecule is prostacyclin.

8. The method of claim 1, wherein the vector is a non-viral vector.

9. The method of claim 1, wherein the stably transfected mammalian cells produce the biomolecule continuously.

10. Stably transfected mammalian cells produced by the method of claim 1 or a pharmaceutical composition thereof.

11. A method for treating a pathophysiological condition associated with at least a decrease in production of a biomolecule in a subject, comprising:
administering to the subject a pharmacological amount of the stably transfected mammalian cells of claim 10, said mammalian cells producing the biomolecule in the subject, thereby treating the pathophysiological condition.

12. The method of claim 11, wherein the stably transfected mammalian cells produce the biomolecule continuously.

13. The method of claim 11, wherein the pathophysiological condition is hypertension, atherosclerosis, peripheral vascular diseases, or congestive heart failure.

14. A method for producing prostacyclin in a cell, comprising:
transfecting a plurality of mammalian cells with a vector containing a cDNA that stably overexpresses a hybrid enzyme effective to metabolize endogenous arachidonic acid to prostacyclin in the stably transfected mammalian cells.

15. The method of claim 14, wherein the vector is a non-viral vector.

16. The method of claim 14, wherein the hybrid enzyme comprises:
a human cyclooxygenase isoform-1 or -2 enzyme;
a human prostacyclin synthase enzyme; and
a helical amino acid sequence shown in SEQ ID NO: 1 linking said enzymes.

17. The method of claim 16, wherein the hybrid enzyme is Trip-cat enzyme-1 or -2.

18. The method of claim 14, wherein the stably transfected mammalian cells produce the biomolecule continuously.

19. A cell line engineered to produce prostacyclin, comprising:
a plurality of mammalian cells having endogenous arachidonic acid stably transformed with a nucleic acid stably overexpressing a human hybrid enzyme effective to metabolize the arachidonic acid to prostacyclin in the engineered cells.

20. The engineered cell line of claim 19, wherein the nucleic acid comprises a vector that further comprises regulatory elements effective to overexpress the nucleic acid.

21. The engineered cell line of claim 19, wherein the vector is a non-viral vector.

22. The engineered cell line of claim 19, wherein the human hybrid enzyme comprises:
a human cyclooxygenase isoform-1 or -2 enzyme;
a human prostacyclin synthase enzyme; and
a helical amino acid sequence shown in SEQ ID NO: 1 linking said enzymes.

23. The engineered cell line of claim 22, wherein the human hybrid enzyme is Trip-cat enzyme-1 or Trip-cat enzyme-2.

24. The engineered cell line of claim 19, wherein said cells produce prostacyclin continuously.

25. A pharmaceutical composition, comprising:
a plurality of the engineered cells of claim 19, said hybrid enzyme comprising a human cyclooxygenase isoform-1 or -2 enzyme and a human prostacyclin synthase enzyme linked with the sequence shown in SEQ ID NO: 1; and
a pharmaceutically acceptable carrier.

26. A method for treating a vascular disease in a subject, comprising:
administering to the subject a pharmacologically effective amount of the engineered cells of claim 25, said engineered cells producing prostacyclin in the subject thereby treating the vascular disease.

27. The method of claim 26, wherein the vascular disease is hypertension, atherosclerosis, peripheral vascular diseases, or congestive heart failure.

28. The method of claim 25, wherein the engineered cells produce prostacyclin continuously.

29. A recombinant human hybrid protein, comprising:
a human cyclooxygenase isoform-1 or -2 enzyme;
a human prostacyclin synthase enzyme; and
a helical amino acid sequence with about 10 residues linking said enzymes.

30. The recombinant human hybrid protein of claim 29, wherein the helical linker has a sequence shown in SEQ ID NO: 1.

31. The recombinant human hybrid protein of claim 29, wherein the protein is Trip-cat enzyme-1 or -2.

32. Nucleic acid encoding a human hybrid protein, comprising:
a) an isolated nucleic acid encoding the human hybrid protein of claim 29;
b) an isolated nucleic acid that hybridizes to isolated DNA of a) above; and
c) an isolated nucleic acid differing from the isolated nucleic acid of a) and b) above in codon sequence due to the degeneracy of the genetic code that encodes the human hybrid protein.

33. A vector comprising the nucleic acid of claim 32 and regulatory elements necessary to stably overexpress said nucleic acid in a cell.

34. The vector of claim 33, wherein the vector is a non-viral vector.
